(19) United States
(12) Patent Application Publication
(10) Pub. No.: US 2023/0399311 A1
(43) Pub. Date: Dec. 14, 2023

(54) POLYMORPHS OF SUBSTITUTED ISOQUINOLINE-BASED RHO KINASE INHIBITORS

(71) Applicants: BioAxone BioSciences, Inc., Boston, MA (US); Neurelis, Inc., San Diego, CA (US)

(72) Inventors: Kenneth M. ROSEN, Milton, MA (US); Matthew D. ABBINANTI, Westford, MA (US); Lisa MCKERRACHER, Boston, MA (US); Stuart MADDEN, San Diego, CA (US)

(73) Assignees: BioAxone BioSciences, Inc., Boston, MA (US); Neurelis, Inc., San Diego, CA (US)

(21) Appl. No.: 18/332,476

(22) Filed: Jun. 9, 2023

Related U.S. Application Data
(60) Provisional application No. 63/350,936, filed on Jun. 10, 2022.

(51) Int. Cl. C07D 401/12

(52) U.S. Cl. CPC C07D 401/12; C07B 2200/13; C07B 2200/05

(57) ABSTRACT

The present disclosure relates to crystalline polymorphs of (R)-1-(1-(isoquinolin-5-ylsulfonyl)piperidin-4-yl)ethan-1-amine, salts thereof, pharmaceutical compositions thereof, pharmaceutical compositions comprising crystalline polymorphs of (R)-1-(1-(isoquinolin-5-ylsulfonyl)piperidin-4-yl)ethan-1-amine, salts thereof and methods of treatment of neurological conditions including cavernous angioma, cerebral aneurysm, stroke, subarachnoid hemorrhage, vasospasm after subarachnoid hemorrhage, traumatic brain injury, spinal cord injury, spinal cord injury stroke, seizures, hereditary hemorrhagic telangiectasis, cerebral arteriovascular malformations, sporadic cavernous angioma lesion, cerebral cavernous malformation, or combinations thereof, using the same.

### CROSS-REFERENCE TO RELATED APPLICATION

This application claims priority to U.S. Provisional Application No. 63/350,936 filed Jun. 10, 2022, which is hereby incorporated by reference in its entirety.

### FIELD

The present disclosure relates generally to crystalline polymorphs of rho kinase inhibitors. The present disclosure specifically relates to polymorphs of the isoquinoline compound NRL-1049, salts thereof, and pharmaceutical formulations comprising crystalline forms of the same.

### BACKGROUND

Rho kinase (ROCK) is a serine/threonine kinase that plays a pivotal role in regulation of the cytoskeleton, motility, and junctional contacts in a variety of tissues. ROCK is activated when the small GTPase Rho (Rho) is activated and plays a key role in complex intracellular signaling cascades. There are two forms of Rho kinase, ROCK1 and ROCK2, both of which are activated by Rho. ROCK1 has widespread tissue distribution, while ROCK2 is expressed primarily in the central nervous system, brain, heart, and lung.

ROCK is known to be abnormally activated in many types of neurotrauma and neurovascular diseases, including but not limited to stroke, subarachnoid hemorrhage, traumatic brain injury, spinal cord injury, and cerebral cavernous malformation. Inhibitors of ROCK promote neurite outgrowth and axon regeneration after injury and are also effective in reducing ROCK activation in endothelial cells after stroke and in neurovascular diseases such as aneurysms and angiomas. ROCK has also been linked to multiple pathologies including, but not limited to cardiovascular disease, immune diseases, metabolic diseases, neurodegenerative diseases, and cancer. Most Rho kinase inhibitors target both ROCK1 and ROCK2 and are thus nonselective. However, nonselective ROCK inhibitors such as Fasudil commonly induce unwanted side effects and are associated with severe complications in cases of long-term use. It has been found that Rho kinase inhibitors that have higher selectivity for ROCK2 decrease the incidence of the associated side effects such as hypotension and can result in better patient outcomes.

There is therefore a need in the field of neurological treatments and therapeutics for high-affinity ROCK inhibitors that are selective for ROCK2.

### SUMMARY

This summary is provided to comply with 37 C.F.R. § 1.73, requiring a summary of the invention briefly indicating the nature and substance of the invention. It is submitted with the understanding that it will not be used to interpret or limit the scope or meaning of the claims.

It has been discovered that the Rho kinase inhibitor compound NRL-1049 targets ROCK2 and has the ability to reverse ROCK activation in brain endothelial cells after neurovascular trauma. Specifically, the (R) enantiomer of NRL-1049 is selective for ROCK2, though the racemic mixture of the (R) and (S) enantiomers is also effective. It has also been discovered that NRL-1049 (R) promotes neurite growth from neurons after a neurological trauma and has the potential to reverse ROCK activation in neurons after trauma in the central nervous system, making this compound an ideal drug candidate for the treatment of neurological conditions.

These discoveries led to the present disclosure, which is directed to NRL-1049 (R), NRL-1049 (S), enantiomeric mixtures, and salts and crystalline polymorphs thereof. Also disclosed are pharmaceutical formulations comprising NRL-1049 (R) and/or NRL-1049 (S) for the treatment of neurotrauma and neurological conditions, including but not limited to cavernous angioma, cerebral aneurysm, stroke, subarachnoid hemorrhage, vasospasm after subarachnoid hemorrhage, traumatic brain injury, spinal cord injury, spinal cord injury stroke, seizures, hereditary hemorrhagic telangiectasis, cerebral arteriovascular malformations, sporadic cavernous angioma lesion, cerebral cavernous malformation, or combinations thereof.

In one aspect, the present disclosure provides one or more salts or crystalline forms of a compound of Formula I, having the structure:

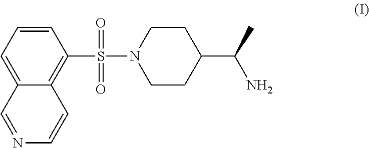 [SMILES: C[C@@H](N)C1CCN(S(=O)(=O)c2cccc3cnccc23)CC1]

In some embodiments, the compound of Formula I is (R)-1-(1-(isoquinolin-5-ylsulfonyl)piperidin-4-yl)ethan-1-amine. The compound of Formula I is also known as NRL-1049 (R) and has been previously referred to as BA-1049 (R). Some embodiments are directed to the adipate salt of the compound of Formula I. The present disclosure also provides pharmaceutical formulations comprising one or more salts or crystalline forms of the Compound of Formula I and methods of using said formulations to treat a neurological condition which may be any of cavernous angioma, cerebral aneurysm, stroke, subarachnoid hemorrhage, vasospasm after subarachnoid hemorrhage, traumatic brain injury, spinal cord injury, spinal cord injury stroke, seizures, hereditary hemorrhagic telangiectasis, cerebral arteriovascular malformations, sporadic cavernous angioma lesion, cerebral cavernous malformation, or combinations thereof. In some embodiments, the compound of Formula I is in the form of a racemic mixture with the compound of Formula II, which has the structure:

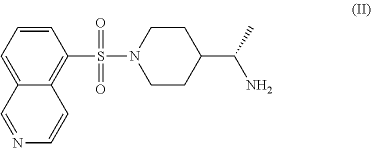 [SMILES: C[C@H](N)C1CCN(S(=O)(=O)c2cccc3cnccc23)CC1]

In some embodiments, the compound of Formula II is (S)-1-(1-(isoquinolin-5-ylsulfonyl)piperidin-4-yl)ethan-1-amine, and is also known as NRL-1049 (S).

Some embodiments further describe Form A, Form B, or Form C of the compound of Formula I, which are crystalline polymorphs of the adipate salt of the compound of Formula I. The adipate salt of the compound of Formula I may also be referred to as NRL-1049 (R) adipate.

There is provided a pharmaceutical formulation comprising a therapeutically effective amount of a crystalline form of a compound of Formula I.

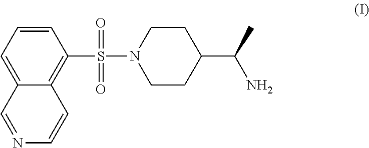 [SMILES: C[C@@H](N)C1CCN(S(=O)(=O)c2cccc3cnccc23)CC1]

In some embodiments, at least 95% of the crystalline form is Form A.

In some embodiments, the crystalline form is a salt comprising a pharmaceutically acceptable counterion.

In some embodiments, the pharmaceutically acceptable counterion is any of hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, tosylate, mesylate, malate, maleate, madelate, gentisinate, fumarate, gluconate, glucaronate, saccharate, formate, benzoate, glutamate, methanesulfonate, ethanesulfonate, benzenesulfonate, p-toluenesulfonate, pamoate, and adipate.

In some embodiments, the pharmaceutically acceptable counterion is adipate.

In some embodiments, the crystalline form is deuterated.

In some embodiments, at least 98% of the crystalline form is Form A.

In some embodiments, at least 99% of the crystalline form is Form A.

In some embodiments, at least 99.5% of the crystalline form is Form A.

In some embodiments, at least 99.9% of the crystalline form is Form A.

In some embodiments, at least 99.99% of the crystalline form is Form A.

In some embodiments, Form A is characterized by an XRPD pattern having a peak expressed in degrees 2θ (±0.2) of about 12.2.

In some embodiments, Form A is characterized by an XRPD pattern having peaks expressed in degrees 2θ (±0.2) of about 12.2 and about 15.9.

In some embodiments, Form A is characterized by an XRPD pattern having peaks expressed in degrees 2θ (±0.2) of about 12.2, about 15.9, and about 19.8.

In some embodiments, Form A is characterized by an XRPD pattern having peaks expressed in degrees 2θ (±0.2) of about 4, about 6, about 12.2, about 15.9, and about 19.8.

In some embodiments, Form A is characterized by a DSC comprising an endothermic event with a peak at about 147° C.

There is provided a crystalline form of a compound of Formula I:

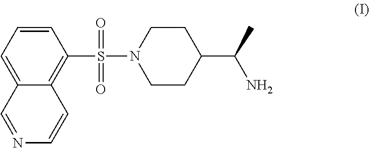 [SMILES: C[C@@H](N)C1CCN(S(=O)(=O)c2cccc3cnccc23)CC1]

In some embodiments, the crystalline form is Form B.

In some embodiments, Form B is characterized by an XRPD pattern having a peak expressed in degrees 2θ (±0.2) of about 17.2.

In some embodiments, Form B is characterized by an XRPD pattern having peaks expressed in degrees 2θ (±0.2) of about 17.2 and about 23.8.

In some embodiments, Form B is characterized by an XRPD pattern having peaks expressed in degrees 2θ (±0.2) of about 17.2, about 23.8, and about 24.3.

In some embodiments, Form B is characterized by an XRPD pattern having peaks expressed in degrees 2θ (±0.2) of about 15, about 17.2, about 20, about 23.8, and about 24.3.

In some embodiments, Form B is characterized by a DSC comprising an endothermic event with a peak at about 91° C.

There is provided a crystalline form of a compound of Formula I:

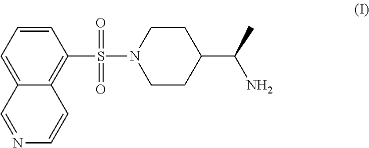 [SMILES: C[C@@H](N)C1CCN(S(=O)(=O)c2cccc3cnccc23)CC1]

In some embodiments, the crystalline form is Form C.

In some embodiments, Form C is characterized by an XRPD pattern having a peak expressed in degrees 2θ (±0.2) of about 21.5.

In some embodiments, Form C is characterized by an XRPD pattern having peaks expressed in degrees 2θ (±0.2) of about 21.5 and about 25.8.

In some embodiments, Form C is characterized by an XRPD pattern having peaks expressed in degrees 2θ (±0.2) of about 21.5, about 25.8, and about 31.2.

In some embodiments, Form C is characterized by an XRPD pattern having peaks expressed in degrees 2θ (±0.2) of about 21.5, about 25.8, about 31.2, and about 38.4.

In some embodiments, Form C is characterized by a DSC comprising an endothermic event with a peak at about 151° C.

There is provided a pharmaceutical formulation comprising a therapeutically effective amount of a crystalline form of a compound of Formula I.

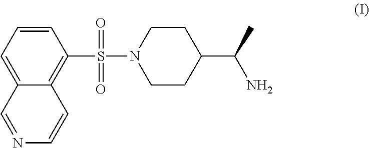 [SMILES: C[C@@H](N)C1CCN(S(=O)(=O)c2cccc3cnccc23)CC1]

In some embodiments, at least 95% of the crystalline form is Form B or Form C.

In some embodiments, the crystalline form is a salt comprising a pharmaceutically acceptable counterion.

In some embodiments, the pharmaceutically acceptable counterion is any of hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, tosylate, mesylate, malate, maleate, madelate, gentisinate, fumarate, gluconate, glucaronate, saccharate, formate, benzoate, glutamate, methanesulfonate, ethanesulfonate, benzenesulfonate, p-toluenesulfonate, pamoate, and adipate.

In some embodiments, the pharmaceutically acceptable counterion is adipate.

In some embodiments, the crystalline form is deuterated.

In some embodiments, at least 98% of the crystalline form is Form B or Form C.

In some embodiments, at least 99% of the crystalline form is Form B or Form C.

In some embodiments, at least 99.5% of the crystalline form is Form B or Form C.

In some embodiments, at least 99.9% of the crystalline form is Form B or Form C.

In some embodiments, at least 99.99% of the crystalline form is Form B or Form C.

There is provided a method for treating a patient having a neurological condition, comprising administering a pharmaceutical formulation which comprises a therapeutically effective amount of a crystalline form of a compound of Formula I.

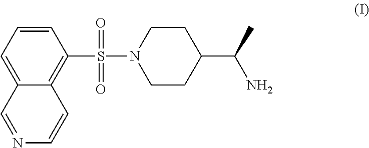 [SMILES: C[C@@H](N)C1CCN(S(=O)(=O)c2cccc3cnccc23)CC1]

In some embodiments, at least 95% of the crystalline form is Form A, Form B, Form C, or combinations thereof.

In some embodiments, at least 98% of the crystalline form is Form A, Form B, Form C, or combinations thereof.

In some embodiments, at least 99% of the crystalline form is Form A, Form B, Form C, or combinations thereof.

In some embodiments, at least 99.5% of the crystalline form is Form A, Form B, Form C, or combinations thereof.

In some embodiments, at least 99.9% of the crystalline form is Form A, Form B, Form C, or combinations thereof.

In some embodiments, at least 99.99% of the crystalline form is Form A, Form B, Form C, or combinations thereof.

In some embodiments, a therapeutically effective amount is from about 0.01 mg/kg to about 1000 mg/kg.

In some embodiments, the pharmaceutical composition is administered orally.

In some embodiments, the pharmaceutical composition is administered once a day.

In some embodiments, the pharmaceutical composition is administered twice a day.

In some embodiments, the neurological condition is selected from cavernous angioma, cerebral aneurysm, stroke, subarachnoid hemorrhage, vasospasm after subarachnoid hemorrhage, traumatic brain injury, spinal cord injury, spinal cord injury stroke, seizures, hereditary hemorrhagic telangiectasis, cerebral arteriovascular malformations, sporadic cavernous angioma lesion, cerebral cavernous malformation, or combinations thereof.

In some embodiments, the neurological condition is a cavernous angioma.

In some embodiments, the patient exhibits an indicator of the neurological condition.

In some embodiments, the indicator is any of symptomatic hemorrhage, recurrent symptomatic hemorrhage, recurrent symptomatic hemorrhage, hemorrhagic stroke, seizures, ataxia, speech and swallowing difficulties, facial paralysis, vision and auditory problems, diaphragmatic spasms, breathing difficulties, headache, fatigue, weakness, tingling, numbness, pain, bladder issues, bowel issues, respiratory distress, double vision, facial droop, balance problems, pupil and vision changes, nausea, projectile vomiting, confusion, impaired consciousness, overactivation of rho signaling in endothelial cells, impaired angiogenesis, defects in vessel-like tube formation, loss of endothelial cell invasion of the extra-cellular matrix, disruption in endothelial barrier integrity, increased intracellular actin stress fibers, decreased intra-endothelial junctions, vascular leakage, increased permeability at intra-endothelial junctions, previous instance of cavernous angioma, or combinations thereof.

In some embodiments, administering the pharmaceutical formulation results in a reduction of one or more symptoms of the neurological condition.

The method of embodiment 51, wherein the indicator is any of symptomatic hemorrhage, recurrent symptomatic hemorrhage, recurrent symptomatic hemorrhage, hemorrhagic stroke, seizures, ataxia, speech and swallowing difficulties, facial paralysis, vision and auditory problems, diaphragmatic spasms, breathing difficulties, headache, fatigue, weakness, tingling, numbness, pain, bladder issues, bowel issues, respiratory distress, double vision, facial droop, balance problems, pupil and vision changes, nausea, projectile vomiting, confusion, impaired consciousness, overactivation of rho signaling in endothelial cells, impaired angiogenesis, defects in vessel-like tube formation, loss of endothelial cell invasion of the extra-cellular matrix, disruption in endothelial barrier integrity, increased intracellular actin stress fibers, decreased intra-endothelial junctions, vascular leakage, increased permeability at intra-endothelial junctions, or combinations thereof.

In some embodiments, administering the pharmaceutical formulation results in an improvement of one or more health parameters of the patient.

In some embodiments, the health parameter of the patient is any of blood brain barrier permeability, lesion stability, modified Rankin Scale, Mini-Mental State Examination, Quality of Life [QoL], 5D, Euro-Quality of Life, Visual Analogue Scale, Neuro-QoL, or any combination thereof.

### BRIEF DESCRIPTION OF THE DRAWINGS

Aspects, features, benefits, and advantages of the embodiments described herein will be apparent with regard to the following description, appended claims, and accompanying drawings where:

FIG. 1 is an ¹H NMR spectrum of Form A of the adipate salt of NRL-1049 (R) in DMSO-d₆.

FIG. 2 is an X-ray powder diffraction spectrum of the crystalline polymorph Form A of NRL-1049 (R) adipate.

FIG. 3 is a differential scanning calorimetry trace for Form A of NRL-1049 (R) adipate.

FIG. 4 is a thermogravimetric analysis spectrum for Form A of NRL-1049 (R) adipate.

FIG. 5 is an ¹H NMR spectrum of Form B of the adipate salt of NRL-1049 (R) in DMSO-d₆.

FIG. 6 is an X-ray powder diffraction spectrum of the crystalline polymorph Form B of NRL-1049 (R) adipate.

FIG. 7 is a differential scanning calorimetry trace for Form B of NRL-1049 (R) adipate.

FIG. 8 is a thermogravimetric analysis spectrum for Form B of NRL-1049 (R) adipate.

FIG. 9 is an ¹H NMR spectrum of Form C of the adipate salt of NRL-1049 (R) in DMSO-d₆.

FIG. 10 is an X-ray powder diffraction spectrum of the crystalline polymorph Form C of NRL-1049 (R) adipate.

FIG. 11 is a differential scanning calorimetry trace for Form C of NRL-1049 (R) adipate.

FIG. 12 is a thermogravimetric analysis spectrum for Form C of NRL-1049 (R) adipate.

FIG. 13 is an overlay of the XRPD spectra of Form A, Form B, and Form C.

FIG. 14 is a line graph illustrating the change in purity of NRL-1049 adipate in each buffer solution over time.

### DETAILED DESCRIPTION

This disclosure is not limited to the particular systems, devices and methods described, as these may vary. The terminology used in the description is for the purpose of describing the particular versions or embodiments only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The transitional term “comprising,” which is synonymous with “including,” “containing,” or “characterized by,” is inclusive or open-ended and does not exclude additional, unrecited elements or method steps.

As used herein, the modifier “about” is intended to have its regularly recognized meaning of approximately. In some embodiments, the term may be more precisely interpreted as meaning within a particular percentage of the modified value, e.g. “about” may in some embodiments mean±20%, ±10%, ±5%, ±2%, or ±1% or less.

As used herein, “consists of” or “consisting of” means that the composition, formulation, or the method includes only the elements, steps, or ingredients specifically recited in the particular claimed embodiment or claim.

A used herein, the term “consisting essentially of” or “consists essentially of” means that the composition, formulation, or the method includes only the elements, steps, or ingredients specifically recited in the particular claimed embodiment or claim and may optionally include additional elements, steps, or ingredients that do not materially affect the basic and novel characteristics of the particular embodiment or claim.

The phrase “pharmaceutically acceptable” refers to those compounds, materials, pharmaceutical compositions, and/or dosage forms that are, within the scope of sound medical judgement, generally regarded as safe and non-toxic. In particular, pharmaceutically acceptable carriers, diluents, or other excipients used the pharmaceutical formulation of this disclosure are physiologically tolerable, compatible with other ingredients, and do not typically produce an allergic or similar untoward reaction when administered to a patient. Preferably, as used herein, the term “pharmaceutically acceptable” means approved by a regulatory agency of the Federal government or a state government or listed in the U.S. Pharmacopoeia or other generally recognized pharmacopoeia for use in animals, and more particularly humans.

The terms “subject,” “individual” or “patient” are used interchangeably and as used herein are intended to include human and non-human animals. Non-human animals include all vertebrates, e.g. mammals and non-mammals, such as non-human primates, sheep, dogs, cats, cows, horses, chickens, amphibians, and reptiles, although mammals are preferred, such as nonhuman primates, sheep, dogs, cats, cows and horses. Preferred subjects include human patients. The methods are particularly suitable for treating human patients having a condition, disease or disorder described herein.

As used herein, the term “therapeutic” means an agent utilized to treat, combat, ameliorate, prevent or improve an unwanted condition or disease of a patient.

A “therapeutically effective amount” of a compound, pharmaceutically acceptable salt thereof or pharmaceutical composition according to any embodiment described herein, is an amount sufficient to produce a selected effect on at least one symptom or parameter of a specific disease or disorder. The therapeutic effect may be objective (i.e., measurable by some test or marker) or subjective (i.e., subject gives an indication of or feels an effect or physician observes a change). The effect contemplated herein, includes both medical therapeutic and/or prophylactic treatment, as appropriate. The specific dose of a compound administered according to this disclosure to obtain therapeutic and/or prophylactic effects is determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the patient; the time of administration, route of administration, and rate of excretion of the specific compound employed and the duration of the treatment; The therapeutically effective amount administered will be determined by the physician in the light of the foregoing relevant circumstances and the exercise of sound medical judgment. A therapeutically effective amount of a compound, according to any embodiment described herein, is typically an amount such that when it is administered in a physiologically tolerable excipient composition, it is sufficient to achieve an effective systemic concentration or local concentration in the tissue.

The terms “treat,” “treated,” or “treating” as used herein, refers to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment.

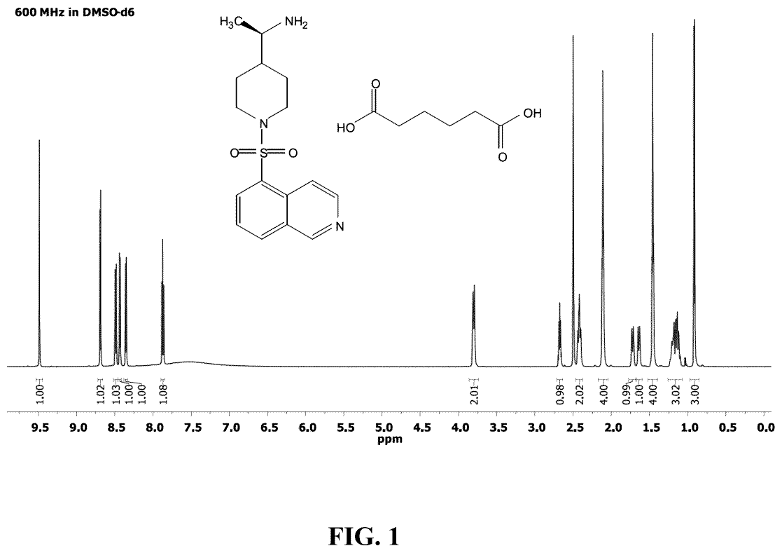

As used herein, an X-ray powder diffraction pattern (also known as XRPD or PXRD) that is “substantially as shown in FIG. 1” or “substantially equivalent to FIG. 1” or the like, relates to an X-ray powder diffraction pattern that would be considered by one skilled in the art to represent a compound possessing the same crystal form as the compound that provided the XRPD pattern of FIG. 1. It is well known and understood to those skilled in the art that the apparatus employed, humidity, temperature, orientation of the powder crystals, and other parameters involved in obtaining an X-ray powder diffraction pattern may cause some variability in the appearance, intensities, and positions of the lines in the diffraction pattern. Thus, an X-ray powder diffraction pattern that is “substantially as shown in FIG. 1” or the like, may not necessarily show each of the lines of any one of the diffraction patterns presented herein, and/or may show a slight change in appearance, intensity, or a shift in position of said lines resulting from differences in the conditions involved in obtaining the data. A person skilled in the art is capable of determining (e.g., by overlaying) if a sample of a crystalline compound has the same form as, or a different form from, a form disclosed herein by comparison of their XRPD patterns.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 compounds refers to groups having 1, 2, or 3 compounds. Similarly, a group having 1-5 cells refers to groups having 1, 2, 3, 4, or 5 compounds, and so forth.

The present disclosure describes crystalline forms of the rho kinase inhibitor NRL-1049 (R). These crystalline forms may be obtained by methods known in the art including recrystallization or evaporation. Different solvents may be used to produce these crystalline forms as would be understood by one of ordinary skill in the art. These crystalline forms may be a salt form of NRL-1049 (R) which comprises a pharmaceutically acceptable counterion such as hydrochloride, hydrobromide, hydroiodide, nitrate, sulfate, bisulfate, tosylate, adipate, mesylate, D-tartrate, L-tartrate, fumarate, malate, maleate, mandelate, succinate, phosphate, acid phosphate, isonicotinate, acetate, lactate, salicylate, citrate, pantothenate, bitartrate, ascorbate, gentisinate, gluconate, glucaronate, saccharate, formate, benzoate, glutamate, methanesulfonate, ethanesulfonate, benzenesulfonate, p-toluenesulfonate, or pamoate.

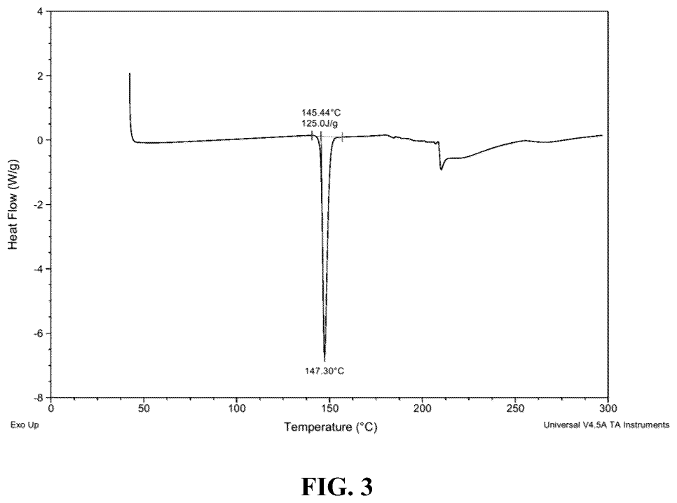

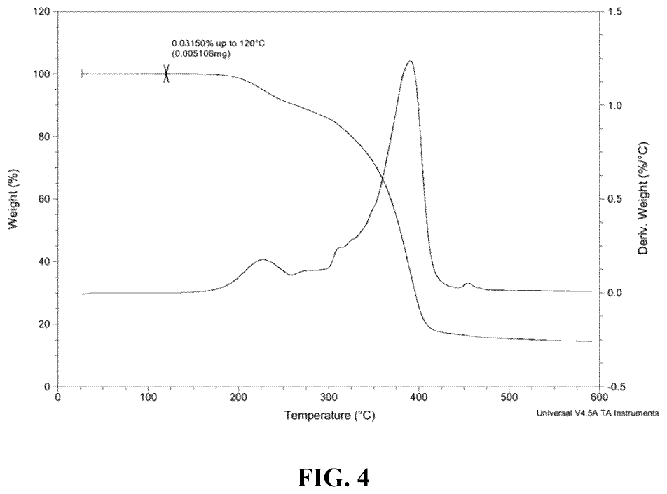

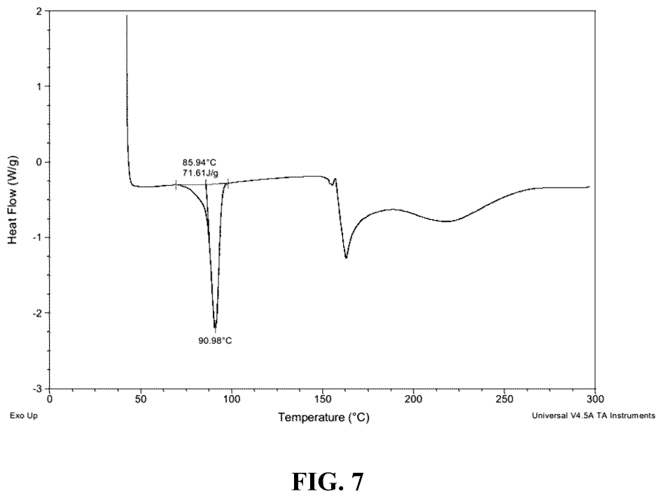

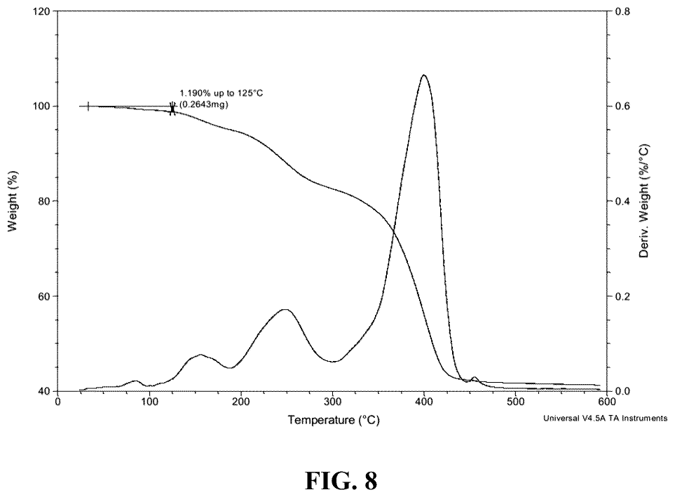

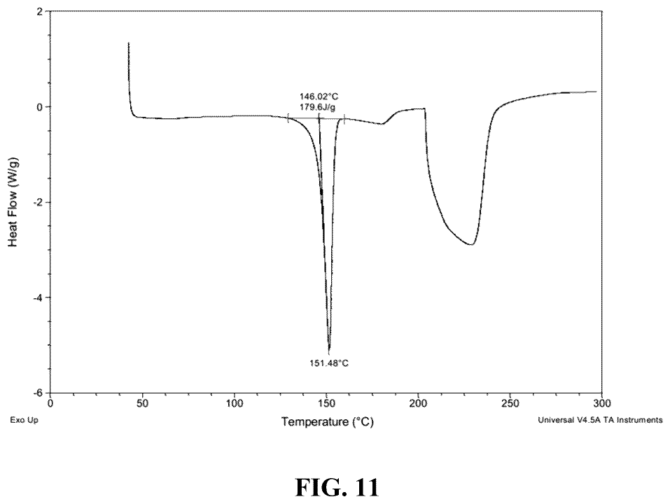

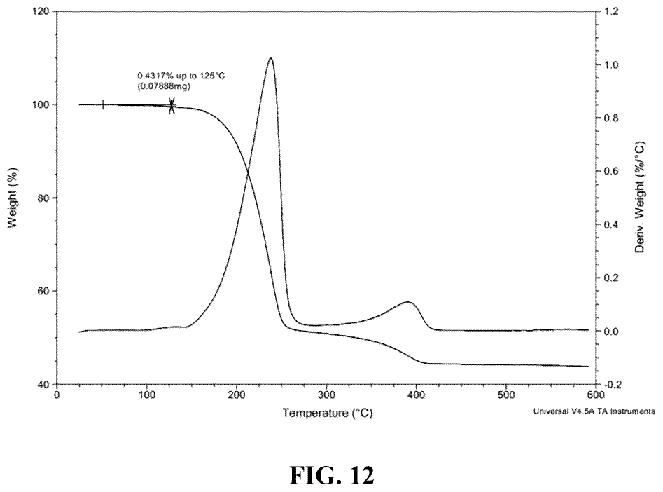

The crystalline forms of NRL-1049 (R) disclosed herein may be polymorphs that are characterized by distinct X-ray powder diffraction patterns. In particular, the polymorph Form A of NRL-1049 (R) adipate may be characterized by an XRPD pattern having peaks expressed in degrees 2θ (±0.2) of about 4, about 6, about 12.2, about 15.9, and about 19.8. Form A of NRL-1049 (R) adipate may also be characterized by a DSC trace that is substantially equivalent to FIG. 3, and a TGA spectrum that is substantially equivalent to FIG. 4. Form B of NRL-1049 (R) adipate may be characterized by an XRPD pattern having peaks expressed in degrees 2θ (±0.2) of about 15, about 17.2, about 20, about 23.8, and about 24.3. Form B of NRL-1049 (R) adipate may be further characterized by a DSC trace that is substantially equivalent to FIG. 7, and a TGA spectrum that is substantially equivalent to FIG. 8. Form C of NRL-1049 (R) adipate may be characterized by an XRPD pattern having peaks expressed in degrees 2θ (±0.2) of about 21.5, about 25.8, about 31.2, and about 38.4. Form B of NRL-1049 (R) adipate may be further characterized by a DSC trace that is substantially equivalent to FIG. 11, and a TGA spectrum that is substantially equivalent to FIG. 12.

Polymorphs of NRL-1049 (R) adipate may be used in pharmaceutical compositions, alone or in combination with pharmaceutically acceptable carriers. The pharmaceutical composition may comprise a therapeutically effective amount of NRL-1049 (R) adipate, either as Form A, Form B, Form C, or in other crystalline, amorphous, or dissolved forms. The pharmaceutical composition may comprise NRL-1049 as a racemic mixture. Said pharmaceutical compositions may be used for the treatment of neurological conditions such as cavernous angioma, cerebral aneurysm, stroke, subarachnoid hemorrhage, vasospasm after subarachnoid hemorrhage, traumatic brain injury, spinal cord injury, spinal cord injury stroke, seizures, hereditary hemorrhagic telangiectasis, cerebral arteriovascular malformations, sporadic cavernous angioma lesion, cerebral cavernous malformation, or combinations thereof.

Patients to which pharmaceutical compositions comprising NRL-1049 (R) adipate are administered may exhibit symptoms or indicators of the neurological condition, including but not limited to symptomatic hemorrhage, recurrent symptomatic hemorrhage, hemorrhagic stroke, seizures, ataxia, speech and swallowing difficulties, facial paralysis, vision and auditory problems, diaphragmatic spasms, breathing difficulties, headache, fatigue, weakness, tingling, numbness, pain, bladder issues, bowel issues, respiratory distress, previous instance of cavernous angioma, or combinations thereof. Administering a pharmaceutical composition comprising NRL-1049 (R) adipate may reduce the instance of one or more of the aforementioned indicators in a patient. Administering a pharmaceutical composition comprising NRL-1049 (R) adipate to a patient may improve one or more health parameters of the patient, wherein the health parameter may be any of blood brain barrier permeability, lesion stability, modified Rankin Scale, Mini-Mental State Examination, Quality of Life [QoL], 5D, Euro-Quality of Life, Visual Analogue Scale, Neuro-QoL, or any combination thereof.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

### EXAMPLES

### Example 1

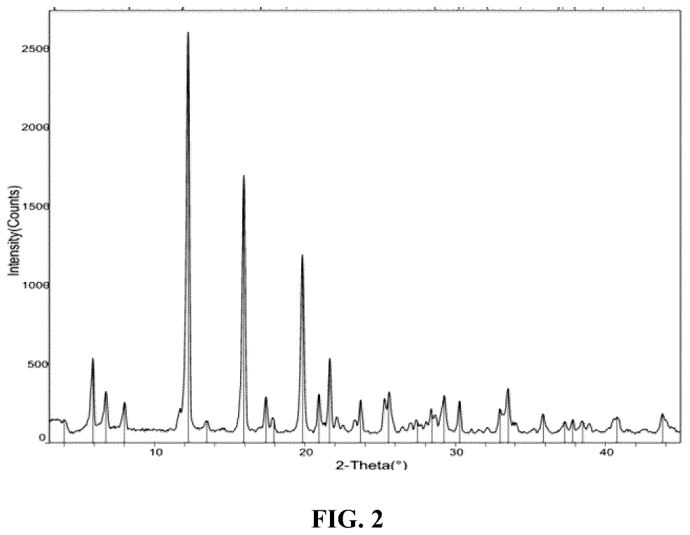

The adipate salt of the compound of Formula I, also known as NRL-1049 (R) adipate, was evaluated by ¹H NMR as shown in FIG. 1 with DMSO-d₆ as the solvent. Compound 1 was further evaluated as crystalline polymorph Form A. An X-ray powder diffraction spectrum (XRPD) of Form A was obtained and is shown in FIG. 2, and further corresponds to the peak report shown in Table 1. It was found that Form A was the predominant crystalline polymorph produced from most solvents and that Form A was maintained through subsequent heating and cooling. Form A was also evaluated by differential scanning calorimetry (DSC), shown in FIG. 3, which showed an endothermic event corresponding to a melt with a peak at 147.30° C. Thermogravimetric analysis (TGA) was also conducted on Form A, and is shown in FIG. 4.

TABLE 1
Peak Report for Form A of NRL-1049 (R) Adipate

| 2θ | d space (Å) | Intensity (%) |
|---|---|---|
| 4.00 | 22.07 | 3.5 |
| 5.90 | 14.96 | 18.2 |
| 6.76 | 13.06 | 9.3 |
| 8.02 | 11.02 | 7.0 |
| 12.24 | 7.22 | 100.0 |
| 13.48 | 6.56 | 2.4 |
| 15.94 | 5.55 | 64.3 |
| 17.42 | 5.09 | 9.0 |
| 17.94 | 4.94 | 3.3 |
| 19.84 | 4.47 | 44.5 |
| 20.94 | 4.23 | 9.4 |
| 21.66 | 4.09 | 18.6 |
| 23.70 | 3.75 | 8.2 |
| 25.56 | 3.48 | 9.1 |
| 27.49 | 3.24 | 1.7 |
| 28.45 | 3.13 | 4.1 |
| 29.28 | 3.04 | 8.8 |
| 30.28 | 2.94 | 7.8 |
| 32.99 | 2.71 | 6.2 |
| 33.52 | 2.67 | 11.3 |
| 35.84 | 2.50 | 5.0 |
| 37.29 | 2.41 | 2.6 |
| 37.82 | 2.37 | 3.4 |
| 38.48 | 2.33 | 2.4 |
| 40.76 | 2.21 | 4.0 |
| 43.79 | 2.06 | 4.8 |

The solubility of Form A of Compound 1 was evaluated at 25° C. and 50° C. in various solvents. Table 2 shows the solubility of Compound 1, NRL-1049 (R) adipate. NRL-1049 (R) adipate exhibited good solubility in chloroform, dimethylformamide, methanol, 2-methoxymethanol, dimethyl sulfoxide, and water at room temperature, and was completely insoluble in heptane at room temperature. It was noted that NRL-1049 adipate was particularly soluble in solvents containing an alcohol group.

TABLE 2
Solubility of NRL-1049 (R)

| | Solubility at | Solubility at |
|---|---|---|
| | 25° C. | 50° C. |
| Solvent | (mg/mL) | (mg/mL) |
| Acetonitrile | <1.0 | <1.0 |
| Chloroform | >50 | >50 |
| Dichloromethane | <1.0 | <1.0 |
| Dimethylformamide | 39.3 | >50 |
| 1,4-Dioxane | 1.5 | 11.9 |
| Methanol | >50 | >50 |
| 2-Methoxyethanol | >50 | >50 |
| Methyl iso-butyl ketone | <1.0 | <1.0 |
| Toluene | <1.0 | <1.0 |
| Tetrahydrofuran | 1.6 | 2.0 |
| Acetone | 15.9 | 45.3 |
| n-Butyl alcohol | 1.6 | 7.5 |
| tert-Butyl methyl ether | <1.0 | <1.0 |
| Dimethyl sulfoxide | >50 | >50 |
| Ethanol | 12.3 | 25.3 |
| Ethyl acetate | <1.0 | <1.0 |
| Ethyl formate | 13.7 | >50 |
| Heptane | 0.0 | 0.0 |
| iso-Butyl acetate | <1.0 | <1.0 |
| iso-Propyl acetate | <1.0 | <1.0 |
| n-Propanol | 1.6 | 23.4 |
| iso-Propanol | 0.5 | 14.7 |
| Water | >50 | >50 |
| Methyl ethyl ketone | <1.0 | 1.5 |
| o-Xylene | <1.0 | <1.0 |
| Propylene glycol | 84 | N/A |
| Polyethylene glycol 300 | 11 | N/A |
| Benzyl alcohol | 156 | N/A |
| Medium chain triglyceride | 0.1 | N/A |
| Sesame oil | 0.04 | N/A |

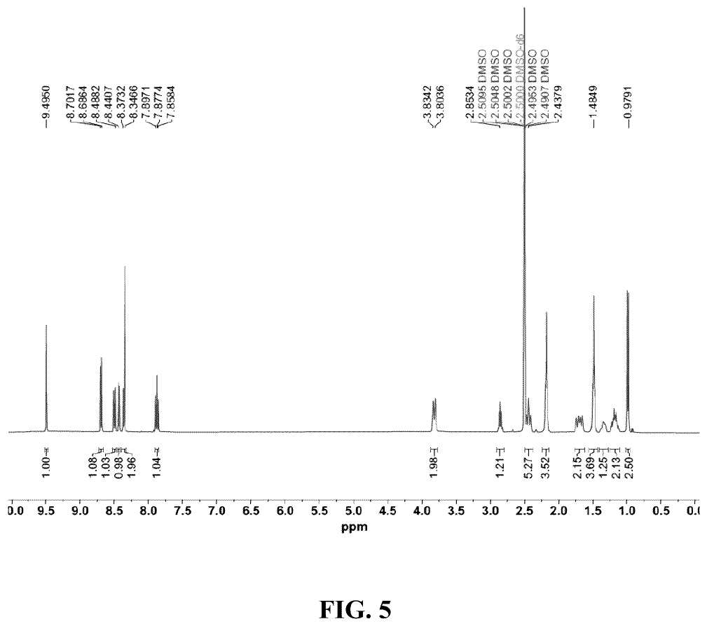

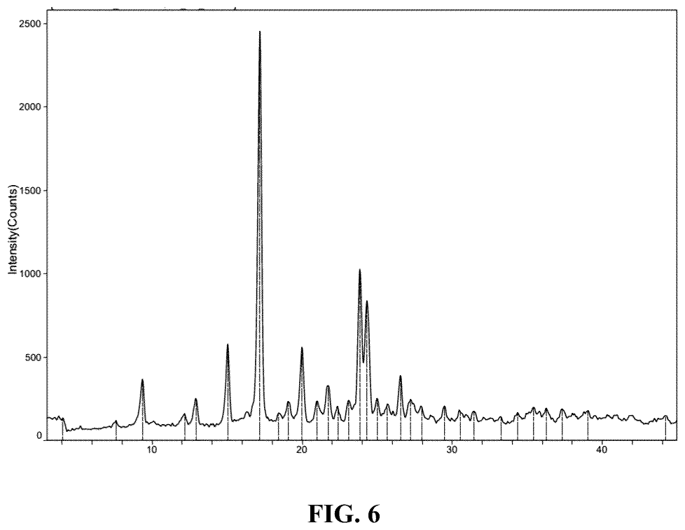

Other polymorphs of NRL-1049 (R) adipate were also identified. Form B is characterized by ¹H NMR in DMSO-d₆ as shown in FIG. 5, and an XRPD spectrum as shown in FIG. 6. The XRPD of Form B is notably distinct from that of Form A, allowing the two polymorphs to be readily distinguished. A peak report for the XRPD of Form B is found in Table 3. Form B was also evaluated by DSC and TGA, which are shown in FIG. 7 and FIG. 8, respectively.

TABLE 3
Peak Report for Form B of NRL-1049 (R) Adipate

| 2θ | d space (Å) | Intensity (%) |
|---|---|---|
| 4.06 | 21.74 | 3.5 |
| 7.59 | 11.62 | 2.0 |
| 9.36 | 9.44 | 12.2 |
| 12.16 | 7.27 | 3.2 |
| 12.92 | 6.84 | 7.3 |
| 15.04 | 5.88 | 20.2 |
| 17.18 | 5.16 | 100.0 |
| 18.44 | 4.81 | 2.0 |
| 19.06 | 4.65 | 5.2 |
| 20.00 | 4.43 | 19.0 |
| 21.00 | 4.22 | 5.2 |
| 21.74 | 4.08 | 9.3 |
| 22.38 | 3.97 | 3.4 |
| 23.10 | 3.84 | 3.6 |
| 23.84 | 3.73 | 38.5 |
| 24.32 | 3.65 | 30.1 |
| 25.00 | 3.55 | 10.7 |
| 25.68 | 3.46 | 3.2 |
| 26.54 | 3.35 | 10.7 |
| 27.24 | 3.27 | 4.8 |
| 27.96 | 3.19 | 3.5 |
| 29.50 | 3.02 | 4.2 |
| 30.53 | 2.92 | 2.4 |
| 31.44 | 2.84 | 2.6 |
| 33.24 | 2.69 | 1.8 |
| 34.36 | 2.61 | 2.1 |
| 35.42 | 2.53 | 3.9 |
| 36.26 | 2.47 | 3.4 |
| 37.32 | 2.41 | 3.2 |
| 39.06 | 2.30 | 2.2 |
| 44.24 | 2.04 | 2.1 |

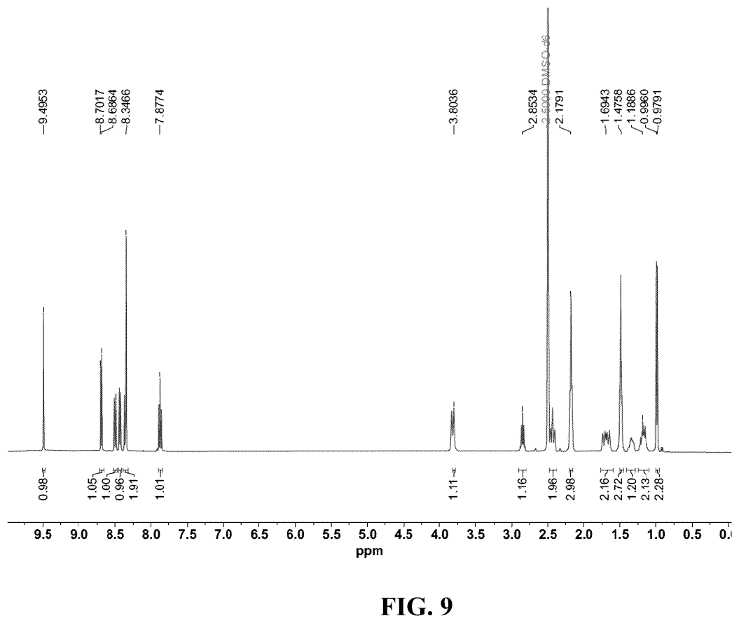

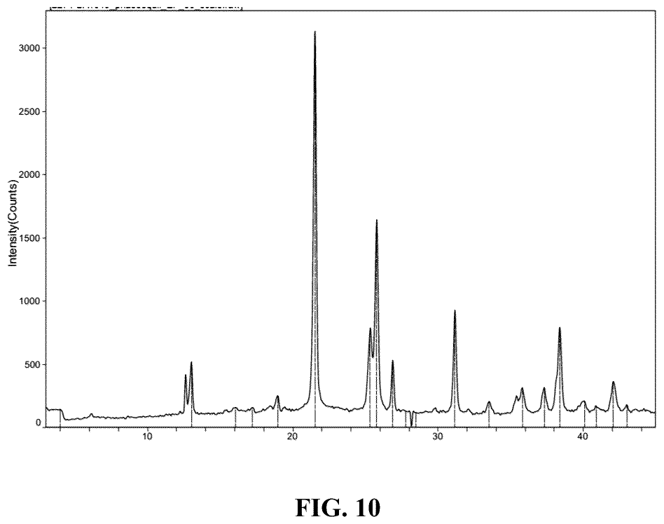

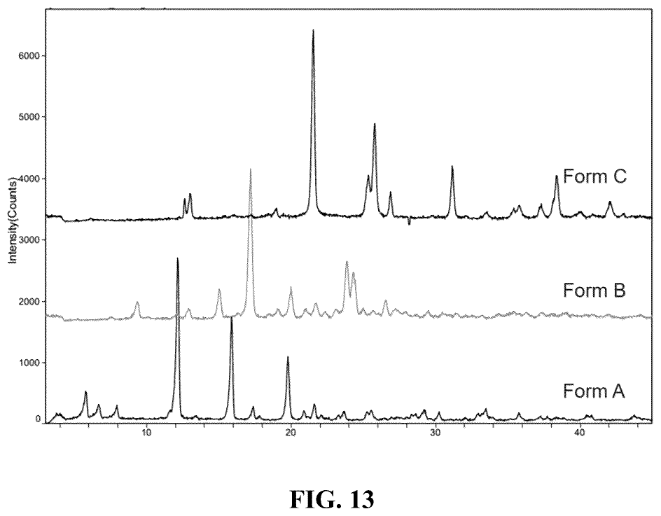

Form C of NRL-1049 (R) adipate was identified. The ¹H NMR spectrum was obtained in in DMSO-d₆ and is shown in FIG. 9. The XRPD of Form C is shown in FIG. 10, and is distinct from that of Form A and Form B. The peak report for the XRPD of Form C can be found in Table 4. The DSC and TGA plots for Form C are shown in FIG. 11 and FIG. 12, respectively. An overlay of the XRPD spectra of each of Form A, Form B, and Form C is shown in FIG. 13.

TABLE 4
Peak Report for Form C of NRL-1049 (R) Adipate

| 2θ | d space (Å) | Intensity (%) |
|---|---|---|
| 4.00 | 22.07 | 2.8 |
| 13.02 | 6.79 | 14.0 |
| 16.04 | 5.52 | 1.6 |
| 17.221 | 5.14 | 1.3 |
| 18.96 | 4.67 | 4.4 |
| 21.54 | 4.12 | 100.0 |
| 25.30 | 3.52 | 20.6 |
| 25.78 | 3.45 | 50.3 |
| 26.88 | 3.31 | 17.4 |
| 27.76 | 3.21 | 3.9 |
| 28.48 | 3.13 | 3.5 |
| 31.16 | 2.87 | 27.4 |
| 33.52 | 2.67 | 3.6 |
| 35.80 | 2.51 | 6.9 |
| 37.32 | 2.40 | 6.4 |
| 38.38 | 2.34 | 22.3 |
| 40.08 | 2.24 | 3.1 |
| 40.89 | 2.20 | 1.6 |
| 42.06 | 2.14 | 8.1 |
| 43.00 | 2.10 | 2.1 |

### Example 2

The partition coefficient of NRL-1049 adipate was determined according to the following procedure. In a 500 mL separation funnel, 120 mL water and 120 mL octanol were combined, mixed well, and allowed to separate into two layers. Approximately 100 mL of the organic phase layer and approximately 100 mL from the aqueous phase layer were removed and utilized in the following steps. Three 100 mL separation funnels were labeled as Sample 1, Sample 2, and Sample 3. To Sample 1 was added 10 mg NRL-1049 adipate, 40 mL aqueous phase, and 10 mL organic phase. To Sample 2 was added 10 mg NRL-1049 adipate, 25 mL aqueous phase, and 25 mL organic phase. To Sample 3 was added 10 mg of NRL-1049 adipate, 10 mL aqueous phase, and 40 mL organic phase. The concentration of NRL-1049 adipate in the aqueous phase and organic phase in each samples was evaluated to determine the partition coefficient P, which is defined as P=(NRL-1049 adipate concentration in organic phase)/(NRL-1049 adipate concentration in aqueous phase). The P values were as follows: Sample 1=0.0053, Sample 2=0.0049, Sample 3=0.0050. It was thus determined that NRL-1049 adipate is highly hydrophilic.

The pKa of NRL-1049 in free base form was measured in triplicate by iterative titration with 1M sodium hydroxide solution. A plot of the pH versus the volume of the titrant was used to calculate the pKa, wherein the pKa was taken as 50% of the inflection point. Table 5 shows the pKa values for each sample and the average pKa of NRL-1049.

TABLE 5
pKa of NRL-1049

| | Sample 1 | Sample 2 | Sample 3 |
|---|---|---|---|
| pKa Measurement #1 | 1.73 | 2.03 | 1.89 |
| pKa Measurement #2 | 1.81 | 2.21 | 1.87 |
| pKa Measurement #3 | 1.83 | 1.82 | 1.98 |
| pKa average | 1.8 | 2.0 | 1.9 |

The pH-dependent solubility of NRL-1049 adipate was also measured. Buffers of pH 1.2 (HCl), pH 4.5 (acetate), and pH 6.8 (phosphate) were prepared. Triplicate samples of NRL-1049 adipate in each buffer were prepared and vortexed and solubility in mg/mL was measured of each. The average solubility of NRL-1049 adipate was found to be 205 mg/mL in pH 1.2 buffer, 328 mg/mL in pH 4.5 buffer, and 178 mg/mL in pH 6.8 buffer.

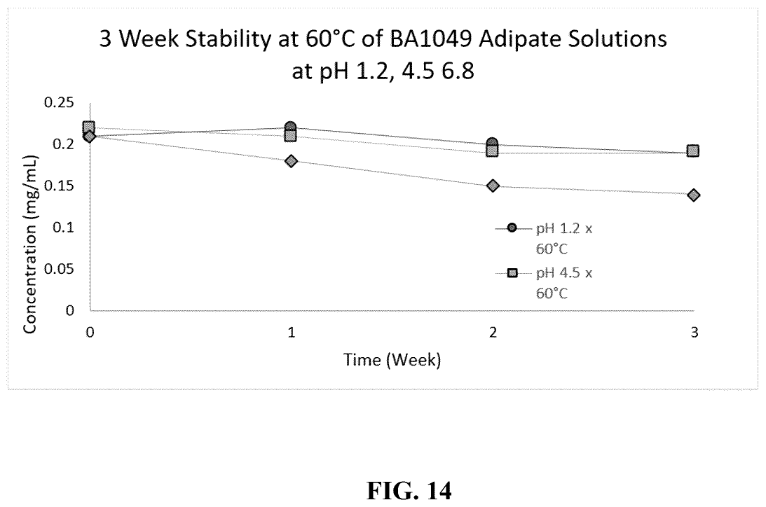

Stability of NRL-1049 adipate as a function of pH was evaluated. Solutions of NRL-1049 adipate in pH 1.2, pH 4.5, and pH 6.8 were prepared. The solutions were stored in vials at 25° C., 40° C., and 60° C. The appearance, pH, assay, and purity were noted at 0, 1, 2, and 3 weeks. Purity was measured by HPLC. These results are summarized in Table 6, below. FIG. 14 illustrates the change in purity of NRL-1049 adipate in each buffer solution over time. As shown in FIG. 14 and in Table 6, NRL-1049 adipate was most stable in the pH 1.2 solution and was least stable in the pH 6.8 buffer.

TABLE 6
pH-Dependent Stability of NRL-1049 Adipate

| | | | | Assay | |
|---|---|---|---|---|---|
| | | | | Recovery (% | |
| | | | | Response/ | |
| Sample | Time/ | | | Time 0 | Purity |
| Buffer | Temperature | Appearance | pH | Response) | (% Area) |
| Hydroxide | Time 0 | Clear | 1.3 | 100 | 99.7 |
| Buffer | | colorless | | | |
| | | solution | | | |
| | 1 Wk, 25° C. | No change | 1.4 | 102.3 | 99.7 |
| | 1 Wk, 40° C. | No change | 1.4 | 102.3 | 99.8 |
| | 1 Wk, 60° C. | No change | 1.3 | 102.1 | 99.6 |
| | 2 Wk, 25° C. | No change | 1.4 | 94.0 | 99.7 |
| | 2 Wk, 40° C. | No change | 1.4 | 94.5 | 99.6 |
| | 2 Wk, 60° C. | No change | 1.3 | 94.3 | 99.6 |
| | 3 Wk, 25° C. | No change | 1.4 | 92.4 | 99.6 |
| | 3 Wk, 40° C. | No change | 1.4 | 91.8 | 99.6 |
| | 3 Wk, 60° C. | No change | 1.3 | 92.8 | 99.6 |
| Acetate | Time 0 | Clear | 4.6 | 100 | 99.8 |
| Buffer | | colorless | | | |
| | | solution | | | |
| | 1 Wk, 25° C. | No change | 4.8 | 98.2 | 99.7 |
| | 1 Wk, 40° C. | No change | 4.7 | 99.2 | 99.7 |
| | 1 Wk, 60° C. | No change | 4.7 | 98.5 | 99.8 |
| | 2 Wk, 25° C. | No change | 4.6 | 89.3 | 99.7 |
| | 2 Wk, 40° C. | No change | 4.5 | 89.7 | 99.7 |
| | 2 Wk, 60° C. | No change | 4.5 | 90.1 | 99.7 |
| | 3 Wk, 25° C. | No change | 4.6 | 88.4 | 99.6 |
| | 3 Wk, 40° C. | No change | 4.5 | 89.1 | 99.7 |
| | 3 Wk, 60° C. | No change | 4.5 | 89.5 | 99.6 |
| Phosphate | Time 0 | Clear | 6.7 | 100 | 99.5 |
| Buffer | | colorless | | | |
| | | solution | | | |
| | 1 Wk, 25° C. | No change | 7.0 | 101.1 | 99.4 |
| | 1 Wk, 40° C. | No change | 7.0 | 94.0 | 95.9 |
| | 1 Wk, 60° C. | No change | 6.9 | 88.3 | 91.3 |
| | 2 Wk, 25° C. | No change | 6.7 | 86.9 | 95.4 |
| | 2 Wk, 40° C. | No change | 6.7 | 87.8 | 94.4 |
| | 2 Wk, 60° C. | No change | 6.7 | 74.4 | 83.0 |
| | 3 Wk, 25° C. | No change | 6.7 | 86.4 | 94.9 |
| | 3 Wk, 40° C. | No change | 6.7 | 78.6 | 91.4 |
| | 3 Wk, 60° C. | No change | 6.8 | 64.9 | 78.8 |

What is claimed is:

1. A pharmaceutical formulation comprising a therapeutically effective amount of a crystalline form of a compound of Formula I: 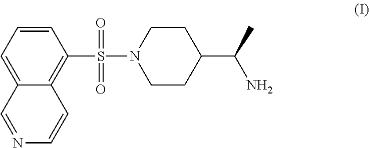 [SMILES: C[C@@H](N)C1CCN(S(=O)(=O)c2cccc3cnccc23)CC1]
wherein at least 95% of the crystalline form is Form A.

2. The pharmaceutical formulation of claim 1, wherein the crystalline form is a salt comprising a pharmaceutically acceptable counterion.

3. The pharmaceutical formulation of claim 2, wherein the pharmaceutically acceptable counterion is any of hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, tosylate, mesylate, malate, maleate, madelate, gentisinate, fumarate, gluconate, glucaronate, saccharate, formate, benzoate, glutamate, methanesulfonate, ethanesulfonate, benzenesulfonate, p-toluenesulfonate, pamoate, and adipate.

4. The pharmaceutical formulation of claim 2, wherein the pharmaceutically acceptable counterion is adipate.

5. The pharmaceutical formulation of claim 1, wherein the crystalline form is deuterated.

6. The pharmaceutical formulation of claim 1, wherein at least 98% of the crystalline form is Form A.

7. The pharmaceutical formulation of claim 1, wherein at least 99% of the crystalline form is Form A.

8. The pharmaceutical formulation of claim 1, wherein at least 99.5% of the crystalline form is Form A.

9. The pharmaceutical formulation of claim 1, wherein at least 99.9% of the crystalline form is Form A.

10. The pharmaceutical formulation of claim 1, wherein at least 99.99% of the crystalline form is Form A.

11. The pharmaceutical formulation of claim 1, wherein Form A is characterized by an XRPD pattern having a peak expressed in degrees 2θ (±0.2) of about 12.2.

12. The pharmaceutical formulation of claim 1, wherein Form A is characterized by an XRPD pattern having peaks expressed in degrees 2θ (±0.2) of about 12.2 and about 15.9.

13. The pharmaceutical formulation of claim 1, wherein Form A is characterized by an XRPD pattern having peaks expressed in degrees 2θ (±0.2) of about 12.2, about 15.9, and about 19.8.

14. The pharmaceutical formulation of claim 1, wherein Form A is characterized by an XRPD pattern having peaks expressed in degrees 2θ (±0.2) of about 4, about 6, about 12.2, about 15.9, and about 19.8.

15. The pharmaceutical formulation of claim 1, wherein Form A is characterized by a DSC comprising an endothermic event with a peak at about 147° C.

16. A crystalline form of a compound of Formula I: 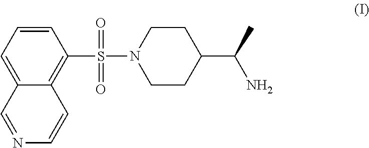 [SMILES: C[C@@H](N)C1CCN(S(=O)(=O)c2cccc3cnccc23)CC1]
wherein the crystalline form is Form B.

17. The crystalline form of claim 16, wherein Form B is characterized by an XRPD pattern having a peak expressed in degrees 2θ (±0.2) of about 17.2.

18. The crystalline form of claim 16, wherein Form B is characterized by an XRPD pattern having peaks expressed in degrees 2θ (±0.2) of about 17.2 and about 23.8.

19. The crystalline form of claim 16, wherein Form B is characterized by an XRPD pattern having peaks expressed in degrees 2θ (±0.2) of about 17.2, about 23.8, and about 24.3.

20. The crystalline form of claim 16, wherein Form B is characterized by an XRPD pattern having peaks expressed in degrees 2θ (±0.2) of about 15, about 17.2, about 20, about 23.8, and about 24.3.

21. The crystalline form of claim 16, wherein Form B is characterized by a DSC comprising an endothermic event with a peak at about 91° C.

22. A crystalline form of a compound of Formula I: 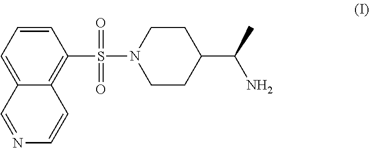 [SMILES: C[C@@H](N)C1CCN(S(=O)(=O)c2cccc3cnccc23)CC1]
wherein the crystalline form is Form C.

23. The crystalline form of claim 22, wherein Form C is characterized by an XRPD pattern having a peak expressed in degrees 2θ (±0.2) of about 21.5.

24. The crystalline form of claim 22, wherein Form C is characterized by an XRPD pattern having peaks expressed in degrees 2θ (±0.2) of about 21.5 and about 25.8.

25. The crystalline form of claim 22, wherein Form C is characterized by an XRPD pattern having peaks expressed in degrees 2θ (±0.2) of about 21.5, about 25.8, and about 31.2.

26. The crystalline form of claim 22, wherein Form C is characterized by an XRPD pattern having peaks expressed in degrees 2θ (±0.2) of about 21.5, about 25.8, about 31.2, and about 38.4.

27. The crystalline form of claim 22, wherein Form C is characterized by a DSC comprising an endothermic event with a peak at about 151° C.

28. A pharmaceutical formulation comprising a therapeutically effective amount of a crystalline form of a compound of Formula I: 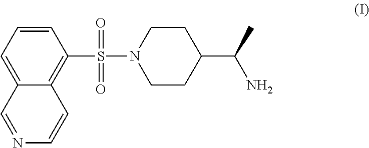 [SMILES: C[C@@H](N)C1CCN(S(=O)(=O)c2cccc3cnccc23)CC1]
wherein at least 95% of the crystalline form is Form B or Form C.

29. The pharmaceutical formulation of claim 28, wherein the crystalline form is a salt comprising a pharmaceutically acceptable counterion.

30. The pharmaceutical formulation of claim 29, wherein the pharmaceutically acceptable counterion is any of hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, tosylate, mesylate, malate, maleate, madelate, gentisinate, fumarate, gluconate, glucaronate, saccharate, formate, benzoate, glutamate, methanesulfonate, ethanesulfonate, benzenesulfonate, p-toluenesulfonate, pamoate, and adipate.

31. The pharmaceutical formulation of claim 29, wherein the pharmaceutically acceptable counterion is adipate.

32. The pharmaceutical formulation of claim 28, wherein the crystalline form is deuterated.

33. The pharmaceutical formulation of claim 28, wherein at least 98% of the crystalline form is Form B or Form C.

34. The pharmaceutical formulation of claim 28, wherein at least 99% of the crystalline form is Form B or Form C.

35. The pharmaceutical formulation of claim 28, wherein at least 99.5% of the crystalline form is Form B or Form C.

36. The pharmaceutical formulation of claim 28, wherein at least 99.9% of the crystalline form is Form B or Form C.

37. The pharmaceutical formulation of claim 28, wherein at least 99.99% of the crystalline form is Form B or Form C.

38. A method for treating a patient having a neurological condition, comprising administering a pharmaceutical formulation which comprises a therapeutically effective amount of a crystalline form of a compound of Formula I: 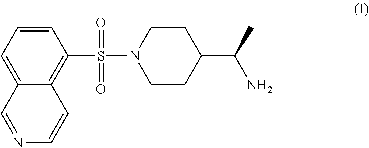 [SMILES: C[C@@H](N)C1CCN(S(=O)(=O)c2cccc3cnccc23)CC1]
wherein at least 95% of the crystalline form is Form A, Form B, Form C, or combinations thereof.

39. The method of claim 38, wherein at least 98% of the crystalline form is Form A, Form B, Form C, or combinations thereof.

40. The method of claim 38, wherein at least 99% of the crystalline form is Form A, Form B, Form C, or combinations thereof.

41. The method of claim 38, wherein at least 99.5% of the crystalline form is Form A, Form B, Form C, or combinations thereof.

42. The method of claim 38, wherein at least 99.9% of the crystalline form is Form A, Form B, Form C, or combinations thereof.

43. The method of claim 38, wherein at least 99.99% of the crystalline form is Form A, Form B, Form C, or combinations thereof.

44. The method of claim 38, wherein a therapeutically effective amount is from about 0.01 mg/kg to about 1000 mg/kg.

45. The method of claim 38, wherein the pharmaceutical composition is administered orally.

46. The method of claim 38, wherein the pharmaceutical composition is administered once a day.

47. The method of claim 38, wherein the pharmaceutical composition is administered twice a day.

48. The method of claim 38, wherein the neurological condition is selected from cavernous angioma, cerebral aneurysm, stroke, subarachnoid hemorrhage, vasospasm after subarachnoid hemorrhage, traumatic brain injury, spinal cord injury, spinal cord injury stroke, seizures, hereditary hemorrhagic telangiectasis, cerebral arteriovascular malformations, sporadic cavernous angioma lesion, cerebral cavernous malformation, or combinations thereof.

49. The method of claim 38, wherein the neurological condition is a cavernous angioma.

50. The method of claim 38, wherein the patient exhibits an indicator of the neurological condition.

51. The method of claim 50, wherein the indicator is any of symptomatic hemorrhage, recurrent symptomatic hemorrhage, recurrent symptomatic hemorrhage, hemorrhagic stroke, seizures, ataxia, speech and swallowing difficulties, facial paralysis, vision and auditory problems, diaphragmatic spasms, breathing difficulties, headache, fatigue, weakness, tingling, numbness, pain, bladder issues, bowel issues, respiratory distress, double vision, facial droop, balance problems, pupil and vision changes, nausea, projectile vomiting, confusion, impaired consciousness, overactivation of rho signaling in endothelial cells, impaired angiogenesis, defects in vessel-like tube formation, loss of endothelial cell invasion of the extra-cellular matrix, disruption in endothelial barrier integrity, increased intracellular actin stress fibers, decreased intra-endothelial junctions, vascular leakage, increased permeability at intra-endothelial junctions, previous instance of cavernous angioma, or combinations thereof.

52. The method of claim 38, wherein administering the pharmaceutical formulation results in a reduction of one or more symptoms of the neurological condition.

53. The method of claim 52, wherein the symptom is any of symptomatic hemorrhage, recurrent symptomatic hemorrhage, recurrent symptomatic hemorrhage, hemorrhagic stroke, seizures, ataxia, speech and swallowing difficulties, facial paralysis, vision and auditory problems, diaphragmatic spasms, breathing difficulties, headache, fatigue, weakness, tingling, numbness, pain, bladder issues, bowel issues, respiratory distress, double vision, facial droop, balance problems, pupil and vision changes, nausea, projectile vomiting, confusion, impaired consciousness, overactivation of rho signaling in endothelial cells, impaired angiogenesis, defects in vessel-like tube formation, loss of endothelial cell invasion of the extra-cellular matrix, disruption in endothelial barrier integrity, increased intracellular actin stress fibers, decreased intra-endothelial junctions, vascular leakage, increased permeability at intra-endothelial junctions, or combinations thereof.

54. The method of claim 38, wherein administering the pharmaceutical formulation results in an improvement of one or more health parameters of the patient.

55. The method of claim 54, wherein the health parameter of the patient is any of blood brain barrier permeability, lesion stability, modified Rankin Scale, Mini-Mental State Examination, Quality of Life [QoL], 5D, Euro-Quality of Life, Visual Analogue Scale, Neuro-QoL, or any combination thereof.
